(19) United States
(12) Patent Application Publication
(10) Pub. No.: US 2011/0201688 A1
(43) Pub. Date: Aug. 18, 2011

(54) N,N'-DI-P-BROMOPHENYL GUANIDINE TREATMENT FOR STROKE AT DELAYED TIMEPOINTS

(75) Inventors: Keith Pennypacker, Wesley Chapel, FL (US); Javier Cuevas, Lutz, FL (US); Jon Antilla, Tampa, FL (US); Michelle Cortes-Salva, Tampa, FL (US)

(73) Assignee: UNIVERSITY OF SOUTH FLORIDA, Tampa, FL (US)

(21) Appl. No.: 13/090,828

(22) Filed: Apr. 20, 2011

Related U.S. Application Data
(63) Continuation of application No. PCT/US2009/061302, filed on Oct. 20, 2009.
(60) Provisional application No. 61/106,814, filed on Oct. 20, 2008.

(51) Int. Cl. A61K 31/155; C07C 279/18; C07C 277/06; A61P 9/10

(52) U.S. Cl. 514/634; 564/238; 564/239

(57) ABSTRACT

1,3 di-o-tolylguanidine (DTG) was examined as anti-stroke drug with a broad therapeutic window. DTG activates sigma 1 and 2 receptors. Administration of DTG at 24 hours post-stroke to rats reduces neurodegeneration by 85%; this is the only pharmacological agent that has been used successfully at this delayed timepoint. Treatment with DTG provides protection of neurons exposed to hypoxia and blocks activation of immune cells that are responsible for delayed neurodegeneration associated with stroke. Disclosed is an altered DTG structure, placing a bromide at the para position to increase tissue penetrance and efficacy. Results show that N,N′-di-p-bromophenyl guanidine protects cultured neurons under hypoxic conditions but is more potent than DTG. Moreover, N,N′-di-p-bromophenyl guanidine is as least as efficacious as DTG in treating rats 24 hours after experimental stroke.

### CROSS REFERENCE TO RELATED APPLICATIONS

This application is a continuation of prior filed International Application, Serial Number PCT/US2009/061302 filed Oct. 20, 2009, which claims priority to U.S. provisional patent application No. 61/106,814 filed Oct. 20, 2008 which is hereby incorporated by reference into this disclosure.

### GOVERNMENT SUPPORT STATEMENT

This invention was made with government support under Grant No. HL072523 awarded by the National Institutes of Health. The Government has certain rights in the invention.

### FIELD OF INVENTION

This invention relates to immune responses. Specifically, the invention is a method of treating stroke and compounds useful in treating stroke.

### BACKGROUND OF THE INVENTION

Stroke is a cerebrovascular injury, and the third leading killer and first cause of disability in the United States, adversely affecting approximately 800,000 Americans every year (American Heart Association, 2009). Recent reports demonstrate that inflammation and neurodegeneration are essential components in limiting expansion of the infarction and are key components to developing new therapies to extend the therapeutic widow of stroke.

When a cerebral embolic stroke occurs, the infarct zone contains two regions associated with ischemic cell death. The center of the infarction or “core” is the area directly affected by the decrease in blood perfusion, and is where the greatest concentration of cell death can be found. Surrounding the core is the penumbra is a region with diminished blood flow, but where collaterals provide some oxygen and nutrients. However, perfusion in the penumbra is sufficiently reduced resulting in arrested physiological function and some degeneration of neurons (Ginsberg, Adventures in the pathophysiology of brain ischemia: penumbra, gene expression, neuroprotection: the 2002 Thomas Willis Lecture. Stroke. 2003 January; 34(1):214-23).

Neuronal death is enhanced by secondary inflammation caused by the immune response in the penumbra. The inflammatory response is primarily from resident activated microglia and infiltrating macrophages, which enter the central nervous system through the degrading blood brain barrier (Stoll, et al. Inflammation and glial responses in ischemic brain lesions. Prog Neurobiol. 1998 October; 56(2):149-71). Reactive astrocytes and microglia exacerbate cerebral inflammation via their production of pro-inflammatory cytokines and chemokines (Trendelenburg and Dirnagl. Neuroprotective role of astrocytes in cerebral ischemia: focus on ischemic preconditioning. Glia. 2005 June; 50(4):307-20). These immune cells, which normally protect the brain via destruction of pathogens and promotion of tissue repair, become over-activated, and further promote the expansion of tissue damage by releasing high levels of nitric oxide (NO), glutamate, tumor necrosis factor-α (TNF-α), and interleukin-1 (IL-1) (Bal-Price and Brown, Inflammatory neurodegeneration mediated by nitric oxide from activated glia-inhibiting neuronal respiration, causing glutamate release and excitotoxicity. J. Neurosci. 2001 Sep. 1; 21(17):6480-91; Heales, et al. Nitric oxide, mitochondria and neurological disease. Biochim Biophys Acta. 1999 Feb. 9; 1410(2):215-28; Hertz, et al. Signaling and gene expression in the neuron-glia unit during brain function and dysfunction: Holger Hydén in memoriam. Neurochem Int. 2001 September; 39(3):227-52).

Intravenous application of recombinant tissue plasminogen activator (tPA), a thrombolytic agent, is the only FDA approved treatment for stroke and has a very limited therapeutic time window that permits only 2-3% of stroke victims to be treated (National Institute of Neurological Disorders and Stroke (NINDS): (1995) N Engl J Med 333 (24): 1581-7). This “clot-buster” must be administered within three hours of stroke onset (Albers, et al. Antithrombotic and thrombolytic therapy for ischemic stroke: the Seventh ACCP Conference on Antithrombotic and Thrombolytic Therapy. Chest. 2004 September; 126(3 Suppl):4835-5125), and can produce possible adverse effects such as hemorrhage and reperfusion damage from oxygen free radicals (Hacke, et al. Thrombolysis in acute ischemic stroke: controlled trials and clinical experience. Neurology. 1999; 53(7 Suppl 4):53-14; Kumura, et al. Generation of nitric oxide and superoxide during reperfusion after focal cerebral ischemia in rats. Am J. Physiol. 1996 March; 270(3 Pt 1):C748-52; Peters, et al. Increased formation of reactive oxygen species after permanent and reversible middle cerebral artery occlusion in the rat. J Cereb Blood Flow Metab. 1998 February; 18(2):196-205). The limitations and adverse effects of tPA have stimulated the search for alternative treatments for stroke.

Two sigma receptor subtypes have been identified on the basis of their pharmacological profile, with the sigma-1 receptor showing high affinity for positive isomer of bezomorphas such as (+)-pentazocine and (+)-SKF-10,047, and the sigma-2 receptor having high affinity for ibogaine (Vilner and Bowen, Modulation of cellular calcium by sigma-2 receptors: release from intracellular stores in human SK—N—SH neuroblastoma cells. J Pharmacol Exp Ther. 2000 March; 292(3):900-11). Only the sigma-1 receptor has been cloned (Hanner, et al. Purification, molecular cloning, and expression of the mammalian sigma1-binding site. Proc Natl Acad Sci USA. 1996 Jul. 23; 93(15):8072-7), but the sigma-2 receptor has been shown to be a separate molecular entity (Langa, et al. Generation and phenotypic analysis of sigma receptor type I (sigma 1) knockout mice. Eur J. Neurosci. 2003 October; 18(8):2188-96).

Sigma receptors are widely distributed throughout the mammalian body, including the brain and spleen. These receptors recognize a diverse array of centrally acting substances including opiates, antipsychotics, antidepressants, phencyclidine (PCP)-related compounds, and neurosteroids (Walker et al., Sigma receptors: biology and function. Pharmacol Rev. 1990 December; 42(4):355-402; Bowen, Sigma receptors: recent advances and new clinical potentials. Pharm Acta Helv. 2000 March; 74(2-3):211-8). While the function of sigma receptors is not well understood, they have been implicated in numerous physiological and pathophysiological processes such as learning and memory (Senda et al., Ameliorating effect of SA4503, a novel sigma 1 receptor agonist, on memory impairments induced by cholinergic dysfunction in rats. Eur J. Pharmacol. 1996 Nov. 7; 315(1):1-10; Hiramatsu et al., Pharmacological characterization of the ameliorating effect on learning and memory impairment and antinociceptive effect of KT-95 in mice. Behav Brain Res. 2006 Feb. 28; 167(2):219-25. Epub 2005 Oct. 11.), movement disorders (Matsumoto et al., Drug specificity of pharmacological dystonia. Pharmacol Biochem Behav. 1990 May; 36(1):151-5), and drug addiction (McCracken et al., Novel sigma receptor ligands attenuate the locomotor stimulatory effects of cocaine. Eur J. Pharmacol. 1999 Jan. 15; 365(1):35-8). Thus far, two sigma receptor subtypes have been identified on the basis of their pharmacological profile, with the sigma-1 receptor showing high affinity for positive isomer of bezomorphas such as (+)-pentazocine and (+)-SKF-10,047, and the sigma-2 receptor having high affinity for ibogaine (Vilner and Bowen, Modulation of cellular calcium by sigma-2 receptors: release from intracellular stores in human SK—N—SH neuroblastoma cells. J Pharmacol Exp Ther. 2000 March; 292(3):900-11). Only the sigma-1 receptor has been cloned (Hanner et al., Purification, molecular cloning, and expression of the mammalian sigma1-binding site. Proc Natl Acad Sci USA. 1996 Jul. 23; 93(15):8072-7), but the sigma-2 receptor has been shown to be a separate molecular entity Langa, et al. Generation and phenotypic analysis of sigma receptor type I (sigma 1) knockout mice. Eur J. Neurosci. 2003 October; 18(8):2188-96).

Dysregulation of intracellular calcium homeostasis greatly contributes to the demise of neurons following an ischemic insult in the central nervous system (Mattson, Apoptotic and anti-apoptotic synaptic signaling mechanisms. Brain Pathol. 2000 April; 10(2):300-12). Elevation of intracellular calcium disrupts plasma membrane function via activation of calcium-sensitive ion channels (Murai et al., Ca2+-activated K+ currents in rat locus coeruleus neurons induced by experimental ischemia, anoxia, and hypoglycemia. J. Neurophysiol. 1997 November; 78(5):2674-81), and triggers biochemical cascades that ultimately promote processes such as proteolysis, lipolysis, production of reactive oxygen species (Mattson, Apoptotic and anti-apoptotic synaptic signaling mechanisms. Brain Pathol. 2000 April; 10(2):300-12) and activation and infiltration of immune cells. Membrane dysfunction produced by ischemia can stimulate multiple plasma membrane calcium fluxes, including Ca²⁺ fluxes attributable to voltage-gated calcium channels and independent of glutamate receptor activation (Nikonenko et al., Inhibition of T-type calcium channels protects neurons from delayed ischemia-induced damage. Mol. Pharmacol. 2005 July; 68(1):84-9. Epub 2005 Apr. 2).

Sigma receptors have been shown to block both voltage-gated calcium channels and ionotropic glutamate receptors (Zhang and Cuevas, Sigma receptors inhibit high-voltage-activated calcium channels in rat sympathetic and parasympathetic neurons. J. Neurophysiol. 2002 June; 87(6):2867-79; Monnet et al., Protein kinase C-dependent potentiation of intracellular calcium influx by sigma1 receptor agonists in rat hippocampal neurons. J Pharmacol Exp Ther. 2003 November; 307(2):705-12. Epub 2003 Sep. 15), both are believed to be involved in the dysregulation of intracellular calcium homeostasis accompanying ischemia. Thus, one of the mechanisms by which sigma receptors may prevent these increases in calcium is via the inhibition of multiple plasma membrane calcium channels.

Sigma 1 and sigma 2 receptors are useful targets for decreasing stroke injury at delayed time points (24 hr post-stroke). Activation of these receptors is responsible for the blockage of Ca2+ influx into the cell (Katnik, et al. Sigma-1 receptor activation prevents intracellular calcium dysregulation in cortical neurons during in vitro ischemia. J Pharmacol Exp Ther. 2006 December; 319(3):1355-65. Epub 2006 Sep. 20).

N,N′-di-o-tolyl guanidine (DTG) is a sigma ligand with high affinity for both sigma 1 and 2 receptors. Activation of both sigma receptors has been seen to result in additive or synergistic neuroprotective and anti-inflammatory effects. DTG administration in MCAO rats reduces infarct size by more than 80% (Ajmo, C. T. Jr.; Sigma receptor activation reduces infarct size at 24 hours after permanent middle cerebral artery occlusion in rats. Curr Neurovasc Res. 2006 May; 3(2):89-98). Activation of sigma receptors using the agent, DTG, can reduce stroke damage by 85% when administered 24 hours after experimental stroke. A search of amination and amidation procedures has been performed for cyclic β-diketones (Shafir, A.; Buchwald, S. Highly selective room-temperature copper-catalyzed C—N coupling reactions. J Am Chem. Soc. 2006 Jul. 12; 128(27):8742-3). However, the toxic and expensive reagents of existing methods are not ideal (Bergfeld et al., U.S. Pat. No. 4,898,978; Katritzky, A.; Rogovoy, B. V. Recent developments in guanylating agents. ARKIVOC 2005 (iv) 49-87). Thus, pharmaceuticals aimed at the deleterious effects of stroke, outside the effective treatment window of tPa are extremely beneficial in the treatment of stroke.

### SUMMARY OF THE INVENTION

The present invention develops new methods for the formation of guanidine structures, allowing DTG analogues with anti-ischemic properties superior to those of DTG. These drugs are tested for drug affinity to both sigma 1 and sigma 2 receptors to improve dosing and diminish adverse effects.

### BRIEF DESCRIPTION OF THE DRAWINGS

For a fuller understanding of the invention, reference should be made to the following detailed description, taken in connection with the accompanying drawings, in which:

FIG. 1 is an illustration of a reaction scheme used to generate disubstituted guanidines.

FIG. 2 is an illustration of a reaction scheme variation in aromatic halide for the CuI catalyzed reactions.

FIG. 3 is a graph showing the nuclear magnetic resonance analysis of the resulting product for ¹H NMR.

FIG. 4 is a graph showing nuclear magnetic resonance analysis of the resulting product for ¹³C NMR.

FIG. 5 is an illustration of the structure of N,N′-di-p-bromophenyl guanidine (p-Br-DPG) as determined by NMR analysis.

FIG. 6 shows inhibition of ischemia-induced increases in [Ca²⁺] by DTG analogues. (A) is a graph of the relative inhibition of the analogues on internal calcium levels, set against DTG. (B) are illustrations of the analogues administered.

FIG. 7 shows inhibition of [Ca²⁺]ᵢ dysregulation in cortical neurons. Concentration-response relationship for p-Br-DPG and DTG inhibition of [Ca²⁺]ᵢ increases evoked by acidosis (pH 6.0). Data points represent mean±SEM (n>50 for all) and lines represent best fit to the data using a Langmuir-Hill equation with K_{d} values of 13.5 and 109.3 and Hill coefficients of 1.2 and 0.9 for p-Br-DPG and DTG, respectively.

FIG. 8 shows inhibition of [Ca²⁺]ᵢ dysregulation in cortical neurons. Concentration-response relationship for p-Br-DPG inhibition of [Ca²⁺]ᵢ increases evoked by chemical ischemia (4 mM azide, 0 glucose). Solid line represents best fit to the data (n>50 for all points) using a Langmuir-Hill equation with a K_{d} value of 2.6 and Hill coefficient of 0.7. Dashed line is fit to the data in FIG. 7, shoing p-Br-DPhG inhibition of acidosis-induced Ca²⁺ elevations and is included for comparison.

FIG. 9 is a graph showing the data for rats after surgery for permanent middle cerebral arterial occlusion. Rats were treated as indicated at 24, 48, and 72 h and sacrificed at 96 hours. Brain sections were stained with Fluoro-Jade to determine infarct volumes.

FIG. 10 is a graph showing microglial migration for 4 hrs at 37° C. Control media or 100 μM ATP were used in the absence and presence of DTG or p-Br-DPG (labeled BDTG). Asterisks indicate a significant difference between ATP (p<0.001, n=5) and ATP+BDTG 20 μM (p<0.01, n=5) from DMEM. Significance was determined by one-way ANOVA followed by post-hoc Bonferroni tests.

### DETAILED DESCRIPTION OF THE PREFERRED EMBODIMENT

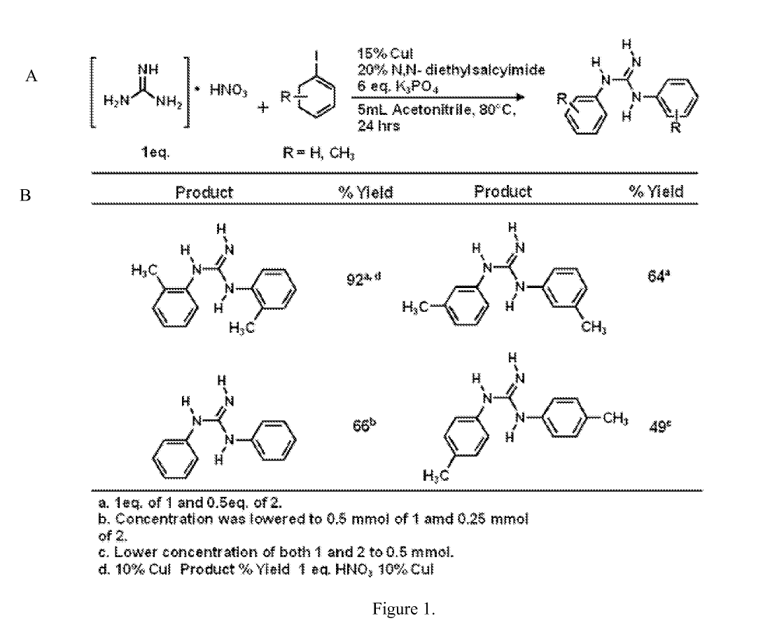

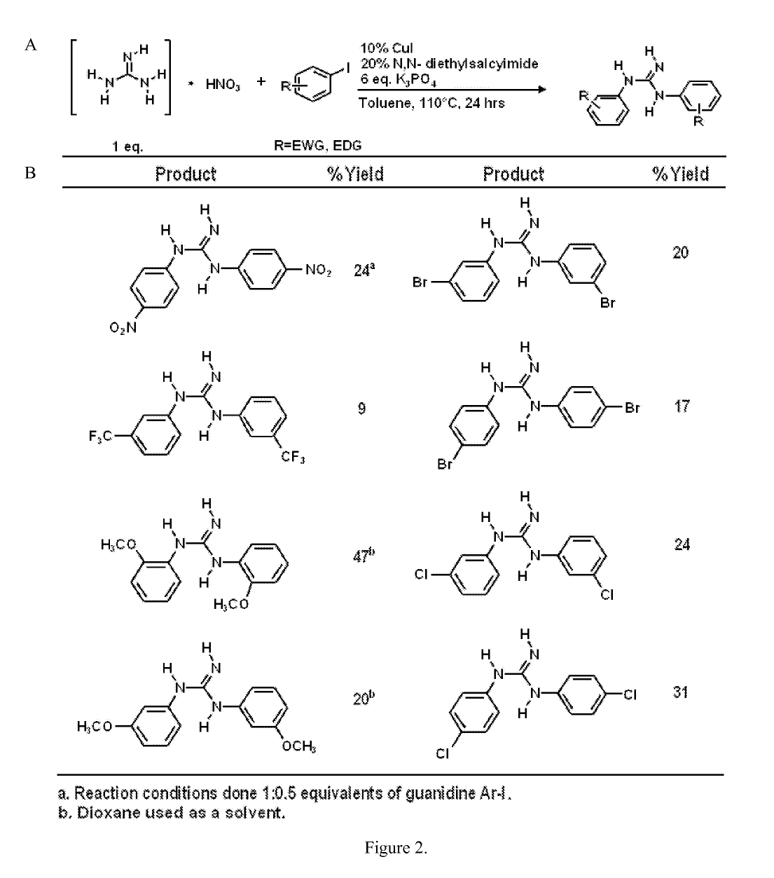

Novel disubstituted guanidines were generated. Alkyl-substituted guanidines were first carried out in flame-dried sealed cap test tube using scheme 1, seen in FIG. 1(a). Alkaline-substituted guanidines were then generated using scheme 2, seen in FIG. 2(a) The resulting solution was stirred for 24 hours, and the product was extracted using dichloromethane, washed with water 3 times, and the organic layer was dried over Na₂SO₄. Yields were calculated for the reaction schemes used, as seen in FIGS. 1(b) and 2(b). The resulting guanidines were found especially useful as an anti-ischemic compound, and shows superior characteristics to DTG.

The term “agonist” refers to a molecule, such as a compound, drug, enzyme activator, or hormone, which enhances the activity of another molecule or the activity of the sigma receptor site.

The term “antagonist” refers to a molecule, such as a compound, drug, enzyme activator, or hormone, which diminishes or prevents the action of another molecule or the activity of the sigma receptor site.

The term “stroke” broadly refers to the development of neurological deficits associated with impaired blood flow to the brain regardless of the cause. Potential causes include, without limiting the scope of the invention, thrombosis, hemorrhage, and embolism. Other injuries may be result in stroke, such as an aneurysm, angioma, blood dyscrasias, cardiac failure, cardiac arrest, septic shock, head trauma, spinal cord trauma, seizure, bleeding from a tumor, or other blood loss.

The term “ischemic” or “ischemic episode” means any circumstance that results in a deficit of blood supply to a tissue, especially the central nervous system (CNS) or brain tissue. When the ischemia is associated with a stroke, it can be either global or focal ischemia. The term “ischemic stroke” means a type of stroke that is of limited extent and caused by a blockage of blood flow. Non-limiting examples include cerebral ischemia, ischemia after cardiac arrest, stroke, multi-faceted dementia, and complications from surgery. Cerebral ischemic episodes result from a deficiency in blood supple to the brain. The spinal cord is also considered part of the CNS, and is equally susceptible to ischemia resulting from diminished blood flow. The term “focal ischemia” is used to refer to a condition resulting from a blockage of a single artery that supplies blood to the brain or spinal cord, resulting in damage to the cells in the territory supplied by that artery. Conversely, “global ischemia” refers to a condition that results from a general diminution of blood flow to the entire tissue, such as the entire brain, forebrain, or spinal cord, thereby causing the death of neurons in selectively vulnerable regions throughout these tissues.

The term “effective amount” or pharmaceutically effective amount” refers to a nontoxic, but significant, amount of the disclosed agent required to provide the desired biological result. The result can be a reduction and/or alleviation of symptoms, causes of disease, or other desired alteration of a biological system. An “effective amount” for therapeutic purposes is the amount of the composition of sigma receptor ligand required to provide a clinically significant decrease in neurodegenerative disease, such as those resulting from ischemic stroke. An appropriate effective amount may be determined by one of ordinary skill in the art using routine experimentation.

The term “treat” or “treatment” means a postponement of progression of a neurodegenerative disease and/or reduction in the severity of symptoms that have or are expected to develop. The term also is intended to include ameliorating the existing neurodegenerative symptoms, preventing symptoms, and ameliorating or preventing the underlying metabolic causes.

The term “patient” includes mammals and non-mammals. Non-limiting examples include humans, non-human primates, species of the family bovidae, species of the family suidae, domestic animals including rabbits, dogs, and cats, laboratory animals, such as rats, mice, guinea pigs, and non-mammals, including birds and fish.

The term “pharmaceutically acceptable salt” means a salt that possesses the desired pharmacological activity of the parent compound. Such salts include, without limiting the scope of the invention, salt derivatives prepared by methods known to those of skill in the art. For example, acid addition salts, formed with inorganic acids, such as hydrochloric acid, hydrobromic acid, sulfuric acid, nitric acid, phosphoric acid, lewis acids, or formed with organic acids, such as acetic acid, propionic acid, hexanoic acid, cyclopentancepropionic acid, glycolica acid, pyruvic acid, lactic acid, malonic acid, succinic acid, malic acid, meleic acid, fumaric acid, and citric acid. Alternatively, the salt derivatives are formed when an acidic poton present in the patent compound is replaced by a metal ion, such as an alkali metal, an alkaline earth ion, or coordinates with an organic base. Some non-limiting exemplary inorganic bases include aluminum hydroxide, calcium hydroxide, potassium hydroxide, sodium carbonate, and sodium hydroxide.

In general, the compounds of the present invention are administered in a therapeutically effective amount by any accepted mode of administration. Suitable dosage ranges depend upon factors known to one skilled in the art. Non-limiting examples of factors include the severity of the disease to be treated, the age of the patient, the relative health of the subject, the potency of the compound utilized, and the route and form of administration. Once of skill in the art will also be capable of ascertaining the therapeutically effective amount of compound needed for a given disease, without undue experimentation and in reliance of his or her experience.

Compound of this invention are administered as pharmaceutical formulations, including those suitable for oral—including buccal and sub-lingual—rectal, nasal, topical, pulmonary, vaginal, or parenteral—including intramuscular, intraarterial, intrathecal, subcutaneous, and intravenous. In some embodiments, intravenous or intraarterial administration is a preferred manner of providing a daily dosing regimen that can be adjusted according to the degree of affliction.

For solid compositions, conventional solid carriers include, without limiting the scope of the invention to any particular material, pharmaceutical grades of mannitol, lactose, starch, magnesium stearate, magnesium carbonate, sodium saccharin, talc, cellulose, glucose, and sucrose. Liquid pharmaceutically administrable compositions can be prepared by dissolving, dispersing, suspending, an active compound of the present invention in an optional pharmaceutical adjuvant or excipient. Non-limiting examples include water, saline, aqueous dextrose, glycerol, ethanol, similar materials, and combinations thereof. If desired, the pharmaceutical composition to be administered may also contain deminimis amounts of nontoxic auxiliary substances, such as wetting or emulsifying agents, pH buffers—such as sodium acetate, sorbitan monolaurate, triethanolamine sodium acetate, triethanolamine oleate. Methods of preparing such substances is known or apparent to one of skill in the art, and described in art-recognized literature, such as Remington's Pharmaceutical Sciences, 18^{th} Ed (Easton, Pa.; Mack Publishing Co., 1990).

Parenteral formulations may be prepared using conventional materials, either as liquid solutions or suspensions, solid forms suitable for use in suspension or solubilization before injection, or emulsion. Injectable solutions or suspensions using known dispersing or wetting agents are known in the art, and optionally include nontoxic diluents or solvents. Exemplary vehicles include, without limiting the scope of the invention, water, Ringer's solution, isotonic sodium chloride, and phosphate buffered saline. Sterile, fixed oils, fatty esters, and polyols. The parenteral solution or solvent may also include a slow release or sustained release systems, which maintains a constant dosage level. Other variations of administration agents containing compounds of the present invention are known in the art, such as embodiments discussed by Oksenberg, et al. (U.S. application Ser. No. 10/868,048)

### Example 1

### The copper-catalyzed addition of 1-bromo-4-iodobenzene to Guanidine Nitrate

The reaction was carried out in flame-dried sealed cap test tubes with magnetic stirring. Copper Iodide, 1-bromo-4-Iodobenzene, and potassium phosphate were purchased from Sigma Aldrich and used as is. N,N-diethylsalycilamide was purchase form Sigma Aldrich and used after standard purification by crystallization or prepared by a known literature procedure (Motoyama, et al. Self-encapsulation of homogeneous catalyst species into polymer gel leading to a facile and efficient separation system of amine products in the Ru-catalyzed reduction of carboxamides with polymethylhydrosiloxane (PMHS). J Am Chem. Soc. 2005 Sep. 28; 127(38):13150-9). Thin layer chromatography was performed on Merck TLC plates (silica gel 60 F254). To a flame-dried reaction tube was added guanidine nitrate (1 mmol, 0.1221 g), 1-bromo-4-iodobenzene (1 mmol, 0.2840 g), CuI (0.1 mmol, 0.190 g), K₃PO₄ (1.2778 g, 6 eq.), N,N-diethylsalycilamide (0.20 mmol, 0.386 g) and toluene (5 mL) was added using a syringe. The addition reactions were run under an inert atmosphere of argon gas. The reaction solvent, toluene was purified by passing through a column of activated alumina under a dry argon atmosphere. After the resulting solution was stirred for 24 hours, the product was extracted using dichloromethane, washed with water 3 times, and the organic layer was dried over Na₂SO₄.

Flash column chromatography was performed with Merck silica gel (230-400 mesh) using ethyl acetate/hexanes/triethylamine=4/1/0.25 to 4/1/0.75 to provide the desired product upon elution. Melting points were determined using a MEL-TEMP 3.0 instrument and are uncorrected. ¹H NMR and ¹³C NMR were recorded on a Bruker Avance DPX-250 (250 MHz) instrument with chemical shifts reported relative to tetramethylsilane (TMS). The HRMS data were measured on ESI/TOF mass spectrometer with electrospray ionization.

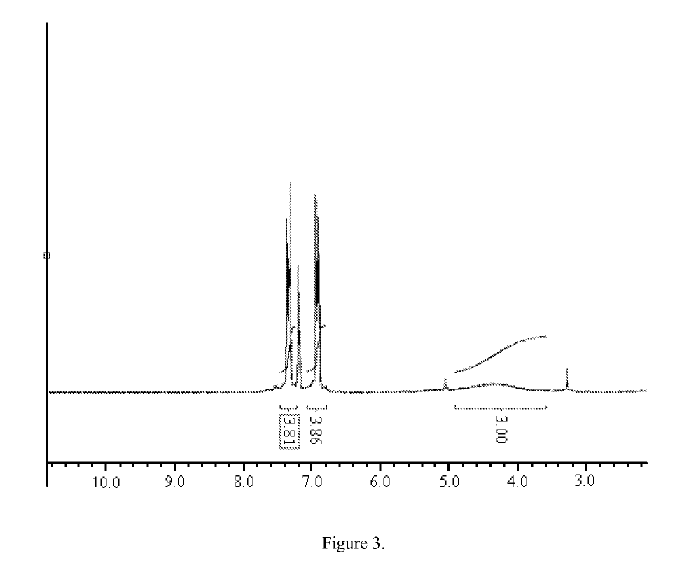

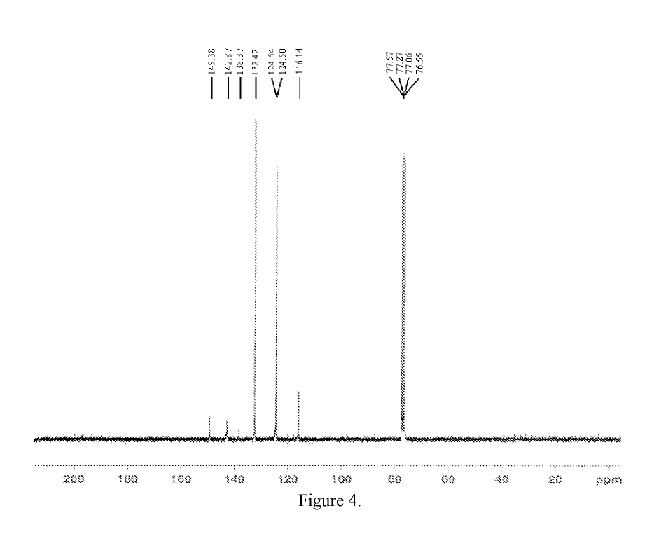

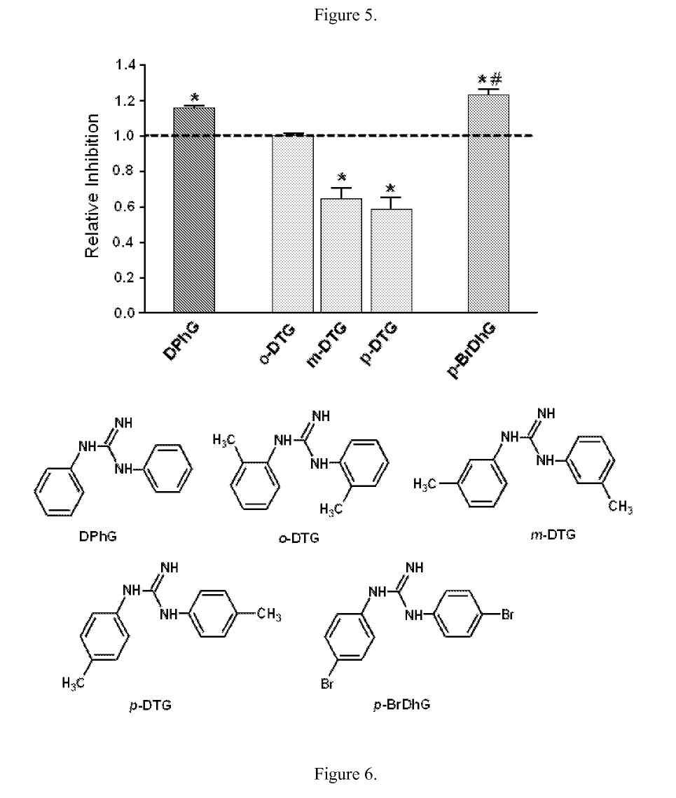

The product analysis showed (hexanes:EtOAc:NEt₃=1:4:0.25 to 4:1:0.75) R_{f}=0.56, as a light pink solid, 34.6 mg, 19% yield. Mp: 169-170° C. ¹H NMR (250 MHz, CDCl₃): δ 4.80 (s, 3H), 6.95 (d, J=8.5 Hz, 4H), 7.30 (d, J=8.5 Hz, 4H). ¹³C NMR (250 MHz, CDCl₃): δ 149.38, 142.87, 138.37, 132.42, 124.80, 124.50, 116.14. MS (ESI): C₁₃H₁₁N₃ Br₂ calculated for ([M+H]⁺) 369.9Found 369.9, seen in FIGS. 3 and 4. The final product was determined to be N,N′-di-p-bromophenyl guanidine (p-Br-DPG), seen in FIG. 5.

### Example 3

### In Vitro Evaluation of DTG Analogues for Calcium Inhibition on Cortical Neuron Cells

The effects of sigma receptors on ischemia-induced changes in intracellular calcium concentrations were studied in cultured cortical neurons from embryonic (E18) rats. Dams were euthanatized by decapitation, uterus removed, and embryos dissected out and placed in isotonic buffer containing 137 mM NaCl, 5 mM KCl, 0.2 mM NaH₂PO₄, 0.2 mM KH₂PO₄, 5.5 mM glucose, 6 mM sucrose (pH 7.4 with NaOH). Cortex were excised and minced, and tissue digested in isotonic buffer containing 0.25% trypsin/EDTA for 10 min at 37° C. and added to 3× volume of high glucose culture media (Dulbecco's Modified Eagle Media; Invitrogen, Inc., Carlsbad, Calif.), 10% (v/v) fetal calf serum, 100 U/ml penicillin and 0.1 mg/ml streptomycin. Cells were counted on a hemocytometer, plated (0.5×10⁶ cells) on 18 mm coverslips coated with poly-1-lysine, and incubated at 37° C. under a 95% air, 5% CO₂ atmosphere. After 24 hr the media was replaced with Neurobasal (Invitrogen, Inc., Carlsbad, Calif.) medium supplemented with B27 (Invitrogen, Inc., Carlsbad, Calif.) and 0.5 mM 1-glutamine to limit astrocyte proliferation in the cultures. Cells were used for studies after 14-21 days in culture.

In vitro ischemia was achieved using the sodium azide/glucose deprivation model, which has been used effectively in numerous studies to mimic in vivo stroke in an in vitro environment, and has been shown to elicit electrophysiological and neurochemical changes that are qualitatively identical to the oxygen/glucose deprivation model of ischemia (Murai et al., Ca²⁺-activated K⁺ currents in rat locus coeruleus neurons induced by experimental ischemia, anoxia, and hypoglycemia. J. Neurophysiol. 1997 November; 78(5):2674-8). The in vitro NaN₃ chemical ischemia disrupts cell metabolism, which ultimately results in plasma membrane dysfunction and intracellular Ca²⁺ dysregulation. Fura-2 dye was used to measure changes in intracellular Ca²⁺ in the cells. Ischemia was induced in the absence or presence of the drugs to test directly the cell response to sigma receptor activation under ischemic conditions. A series of DTG analogues were examined to determine their effect on ischemia-induced [Ca²⁺]ᵢ, as seen in FIGS. 6(A) and (B). The level of resulting [Ca²⁺]ᵢ inhibition was compared to DTG, which was set to a relative [Ca²⁺]ᵢ, level of 1 as indicated by the dashed line.

Electron donating groups gave the best results for the CuI catalyzed cross coupling reactions. The electron withdrawing p-BrDPhG along with DPhG, which limits steric hindrance, were ˜25% more effective than DTG in depressing Ca²⁺ dysregulation in the cells. Short and long term evaluation will be performed in rats after finding the compounds with the highest affinity to the sigma receptors on the neuron cortical cells. Data suggest that both sterics and electronic effects play an important role in the ability of the guanidine structure to regulate ischemia-evoked changes in cell Ca²⁺.

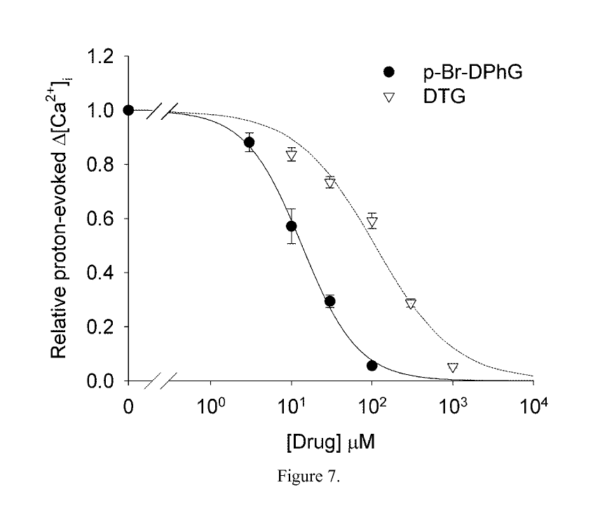

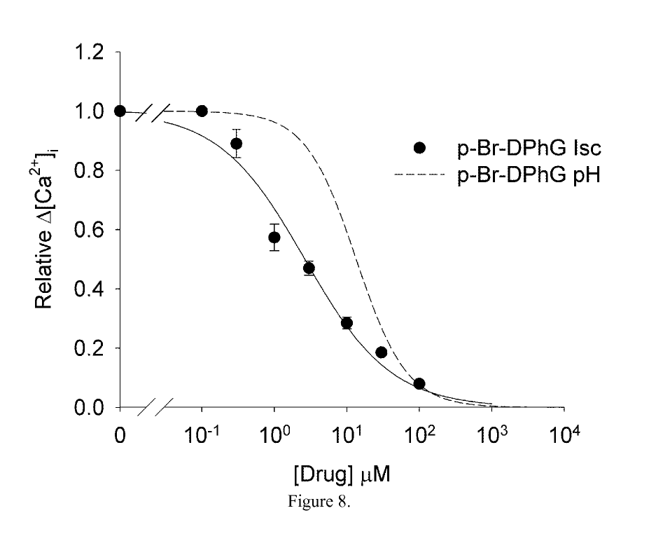

Cerebral tissue acidosis following ischemia or traumatic brain injury has been shown to contribute to cytotoxic brain edema formation. (Ringel F, et al. Lactacidosis-induced glial cell swelling depends on extracellular Ca²⁺. Neurosci Lett. 2006 May 8; 398(3):306-9. Epub 2006 Feb. 15). p-Br-DPG was characterized against DTG to determine the ability to inhibit [Ca²⁺]ᵢ dysregulation in cortical neurons. Cells were administered the indicated amounts of either p-BrDPhG or DTG, followed by cellular [Ca²⁺]ᵢ dysregulation stimulation by acidosis (pH 6.0). Use of p-BrDPhG had a significantly higher effect against [Ca²⁺]ᵢ than DTG, as seen in FIG. 7. The ability of p-Br-DPG to inhibit [Ca²⁺]ᵢ increases evoked by chemical ischemia (4 mM azide, 0 glucose), was then tested, as seen in FIG. 8. A dashed line is superimposed, showing the acidosis results from FIG. 7 for comparison. As can be seen from the Figures, p-BrDPhG is efficient at preventing intracellular calcium increases following neuronal insults, even above the protection afforded by DTG.

### Example 2

### In Vivo Evaluation of DTG Analogues

11 adult male Sprague-Dawley rats (Harlan, Indianapolis, Ind.) weighing 300 to 350 g were housed in a climate controlled room with water and laboratory chow available ad libidum. Sprague-Dawley rats (300-350 g) were randomly assigned to 1 of 3 groups: MCAO (n=4); MCAO and DTG (n=3); or MCAO and p-Br-DPG (n=4). MCAO surgery was performed as previously reported by Vendrame et al. (Vendrame, et al., Infusion of human umbilical cord blood cells in a rat model of stroke dose-dependently rescues behavioral deficits and reduces infarct volume. Stroke. 2004 October; 35(10):2390-5. Epub 2004 Aug. 19) and originally described by Longa et al. (Longa, et al., Reversible middle cerebral artery occlusion without craniectomy in rats. Stroke. 1989 January; 20(1):84-9). Laser Doppler Radar (LDR) was used to monitor decrease in blood perfusion that indicates successful occlusion (Moor Instruments Ltd, Devon, England). A 2 mm diameter hole was drilled into the right parietal bone (1 mm posterior and 4 mm lateral from bregma), and a guide screw was set. The LDR probe (MP10M200ST; Moor) was inserted into the guide screw, and the tip of the probe was placed against the pial surface of the brain. Rats that did not show>55% reduction in perfusion during MCAO were excluded from the study because they generally failed to exhibit infarct damage. For MCAO, the embolus (4 cm long, 6 lb test monofilament) was advanced up the internal carotid artery into the middle cerebral artery and tied off at the internal/external carotid junction to produce permanent occlusion. The rat was then sutured, given a 1 ml subcutaneous injection of saline, and allowed to wake in a fresh cage. All rats received daily injections of 0.04 ml of ketophen and 1 ml of saline.

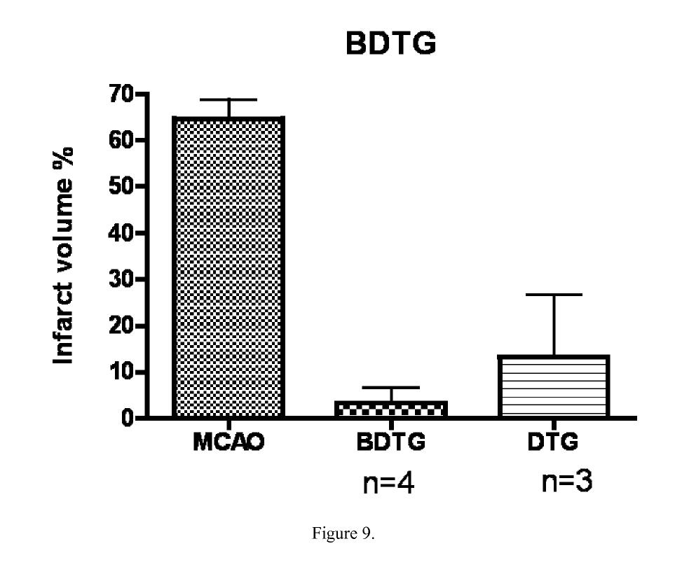

Rats were treated as indicated at 24, 48, and 72 hours after MCAO with vehicle (MCAO), 7.5 mg/kg N,N′-di-p-bromophenyl guanidine (p-Br-DPG) or 7.5 mg/kg 1,3 di-o-tolylguanidine (DTG). The rats were then euthanized at 96 hours. The brains were harvested, fixed in paraformaldehyde, immersed in serial solutions of 20% and 30% sucrose, and sliced into 30 μm sections. After fixation, brains were sectioned and brain sections were stained with Fluoro-Jade. Stroke-induced damage was quantified using the Fluoro-Jade fluorescence to determine infarct volumes. The p-Br-DPG treatment significantly reduced infarct volume (p<0.001) relative to the MCAO group and was slightly more efficacious than DTG treatment, as seen in FIG. 9.

### Example 3

### The Microglial Migratory Response to Chemoattractant Application is Suppressed by DTG and P-Br-DPG

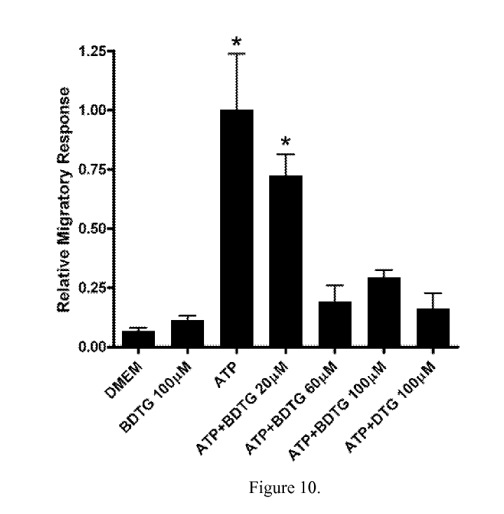

Microglial migration was assayed using a Boyden chamber fitted with a polycarbonate membrane containing 8 μm pores. Microglia (500,000 cells) were placed in the upper chamber and control media or 100 μM ATP were added in the absence and presence of DTG, or various indicated concentrations of p-Br-DPG to the bottom chamber. Microglia were allowed to migrate for 4 hrs at 37° C., and were subsequently stained with DAPI and counted. The addition of ATP significantly increased the migration of microglia compared to DMEM, as seen in FIG. 10. The addition of low-concentration p-Br-DPG (20 μM) also significantly increased microglia migration, whereas higher p-Br-DPG concentrations or DTG inhibited the migration. Further, the amount of microglial migration for p-Br-DPG-treated samples was comparable to DTG-treated samples, indicating that p-Br-DPG is effective in reducing neural immune response, and providing protection after stroke.

In the preceding specification, all documents, acts, or information disclosed do not constitute an admission that the document, act, or information of any combination thereof was publicly available, known to the public, part of the general knowledge in the art, or was known to be relevant to solve any problem at the time of priority.

The disclosures of all publications cited above are expressly incorporated herein by reference, each in its entirety, to the same extent as if each were incorporated by reference individually.

While there has been described and illustrated specific embodiments of a compositions and methods for treatment of stroke, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall therebetween.

What is claimed is:

1. A composition comprising the formula: 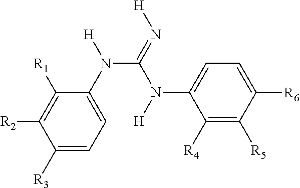
wherein R₁ is selected from the group consisting of OCH₃;
wherein R₂ is selected from the group consisting of Cl, Br, CF₃, NO₂;
wherein R₃ is selected from the group consisting of Cl, Br, CH₃;
wherein R₄ is selected from the group consisting of OCH₃;
wherein R₅ is selected from the group consisting of Cl, Br, CF₃, NO₂; and
wherein R₆ is selected from the group consisting of Cl, Br, CH₃;
or a salt thereof.

2. The composition of claim 1, wherein the composition is selected from the group consisting of 1,3-bis(2-methoxyphenyl)guanidine, 1,3-bis(3-chlorophenyl)guanidine, 1,3-bis(4-chlorophenyl)guanidine, 1,3-bis(3-bromophenyl)guanidine, 1,3-bis(4-bromophenyl)guanidine, and 1,3-bis[3-(trifluoromethyl)phenyl]guanidine, and salts thereof.

3. The composition of claim 2, wherein the composition is 1,3-bis(3-bromophenyl)guanidine.

4. A method of manufacturing a compound of claim 1, comprising:
combining guanidine nitrate, 1-bromo-4-iodobenzene, CuI, K₃PO₄ and, N,N-diethylsalycilamide;
adding a solvent to the combined mixture;
providing an inert gas atmosphere around the reaction;
stirring the resulting solution for 24 hours; and
extracting the product.

5. The method of claim 4, wherein the inert gas is argon.

6. The method of claim 4, wherein the product is extracted using dichloromethane.

7. The method of claim 4, further comprising washing the product with water.

8. The method of claim 4, further comprising drying the product over Na₂SO₄.

9. The method of claim 4, wherein the solvent is toluene.

10. A method of treating stroke, comprising administering at least one compound of claim 1 or a salt thereof.

11. The method of claim 10, wherein the at least one compound was administered within 3 hours of stroke.

12. The method of claim 10, wherein the at least one compound was administered between 3 hours and 6 hours of stroke.

13. The method of claim 10, wherein the at least one compound is 1,3-bis(3-bromophenyl)guanidine.

14. The claim 13, wherein the is 1,3-bis(3-bromophenyl)guanidine is administered between 1 mg/kg and 3 mg/kg.
